(19) United States
(12) Patent Application Publication
(10) Pub. No.: US 2021/0297606 A1
(43) Pub. Date: Sep. 23, 2021

(54) MEDICAL IMAGE PROCESSING DEVICE AND MEDICAL OBSERVATION SYSTEM

(71) Applicant: Sony Olympus Medical Solutions Inc., Tokyo (JP)

(72) Inventor: Takaaki YAMADA, Tokyo (JP)

(73) Assignee: Sony Olympus Medical Solutions Inc., Tokyo (JP)

(21) Appl. No.: 17/179,428

(22) Filed: Feb. 19, 2021

(30) Foreign Application Priority Data
Mar. 18, 2020 (JP) 2020-048141

(51) Int. Cl. H04N 5/243; G09G 5/10; H04N 5/262; A61B 90/20; A61B 5/00; A61B 1/045; A61B 1/00

(52) U.S. Cl. CPC H04N 5/243; G09G 5/10; H04N 5/2628; A61B 90/20; A61B 90/25; A61B 1/045; A61B 1/00045; G09G 2320/0686; G09G 2380/08; A61B 5/0071

(57) ABSTRACT

A medical image processing device includes: a circuitry configured to acquire a captured image obtained by capturing an image of a subject; specify an area of interest among a plurality of image areas in the captured image; and adjust a brightness index value which is an index of brightness for each pixel in the captured image in order to emphasize the area of interest in the plurality of the image areas with respect to other areas. The brightness index value is an index value used for controlling emission brightness of each light emitting element arranged in each of a plurality of divided areas of a display screen in a display device for displaying the captured image.

### CROSS-REFERENCE TO RELATED APPLICATION

This application claims priority from Japanese Application No. 2020-048141, filed on Mar. 18, 2020, the contents of which are incorporated by reference herein in its entirety.

### BACKGROUND

The present disclosure relates to a medical image processing device and a medical observation system.

In the related art, a display device including a transmissive liquid crystal panel and a backlight device that irradiates light from a back surface of the liquid crystal panel is known (see, for example, JP 2020-27273 A).

The display device described in JP 2020-27273 A employs a technique called so-called local dimming that controls emission brightness of each of the light emitting elements arranged for each area of a display screen divided into a plurality of areas, based on a maximum value, an average value, or the like of an input gradation value for each pixel of an input image.

### SUMMARY

By the way, in an operation using a surgical microscope that magnifies and captures an image of a specific visual field area of a subject, an operator performs the operation while observing an image captured by the surgical microscope and displayed on a display device. Here, within all the image areas in the captured image, there is an area of interest that the operator is particularly interested in. That is, if the area of interest is highlighted with respect to other areas, the captured image becomes an image suitable for observation for the operator.

In the display device described in JP 2020-27273 A, a high-contrast captured image may be displayed by giving each light emitting element a brightness difference according to a contrast difference in the input captured image. However, the captured image is not an image in which the above-mentioned area of interest is highlighted with respect to other areas.

Therefore, there is a need for a medical image processing device and a medical observation system capable of generating an image suitable for observation.

According to one aspect of the present disclosure, there is provided a medical image processing device including: a circuitry configured to acquire a captured image obtained by capturing an image of a subject; specify an area of interest among a plurality of image areas in the captured image; and adjust a brightness index value which is an index of brightness for each pixel in the captured image in order to emphasize the area of interest in the plurality of the image areas with respect to other areas, wherein the brightness index value is an index value used for controlling emission brightness of each light emitting element arranged in each of a plurality of divided areas of a display screen in a display device for displaying the captured image.

### BRIEF DESCRIPTION OF THE DRAWINGS

FIG. 1 is a view illustrating a medical observation system according to a first embodiment;

FIG. 2 is a block diagram illustrating the medical observation system;

FIG. 3 is a flowchart illustrating an operation of the medical observation system;

FIGS. 4A, 4B, 4C and 4D are diagrams illustrating the operation of the medical observation system;

FIGS. 5A, 5B, 5C and 5D are diagrams illustrating an operation of a medical observation system according to a second embodiment;

FIGS. 6A, 6B, 6C and 6D are diagrams illustrating an operation of a medical observation system according to a third embodiment;

FIGS. 7A, 7B, 7C and 7D are diagrams illustrating an operation of a medical observation system according to a fourth embodiment;

FIG. 8 is a block diagram illustrating a medical observation system according to a fifth embodiment;

FIG. 9 is a diagram illustrating a spectrum of light emitted from a light source device;

FIG. 10 is a flowchart illustrating the operation of the medical observation system;

FIGS. 11A, 11B, 11C and 11D are diagrams illustrating the operation of a medical observation system;

FIG. 12 is a block diagram illustrating a medical observation system according to a sixth embodiment;

FIG. 13 is a flowchart illustrating the operation of the medical observation system;

FIGS. 14A, 14B, 14C and 14D are diagrams illustrating the operation of the medical observation system;

FIG. 15 is a view illustrating a medical observation system according to a seventh embodiment; and

FIG. 16 is a view illustrating a medical observation system according to an eighth embodiment.

### DETAILED DESCRIPTION

Hereinafter, a mode (hereinafter, “embodiment”) for carrying out the present disclosure will be described with reference to the accompanying drawings. Note that the present disclosure is not limited to embodiments to be described below. Furthermore, in the drawings, the same components are denoted with the same reference numerals.

### First Embodiment

Outline Configuration of Medical Observation System

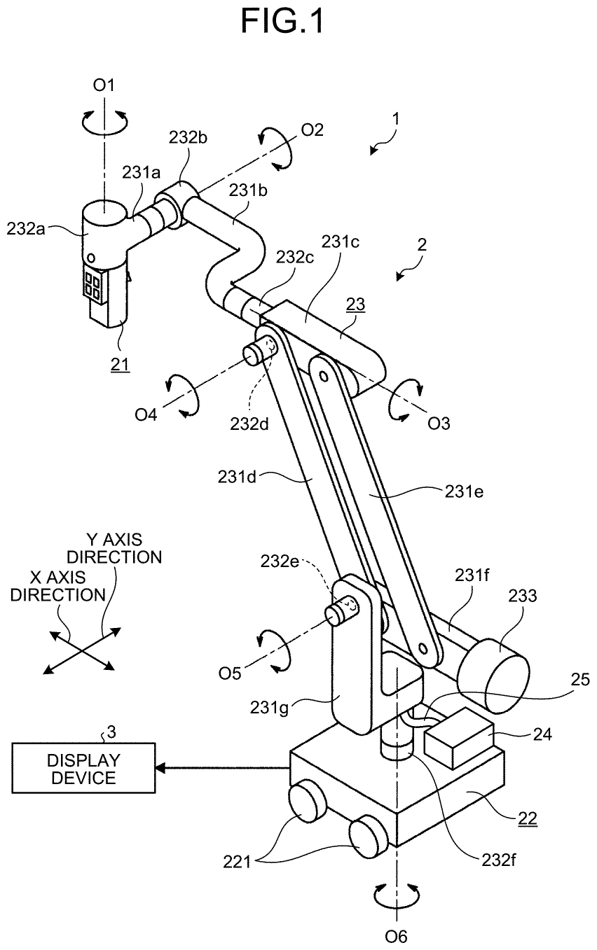

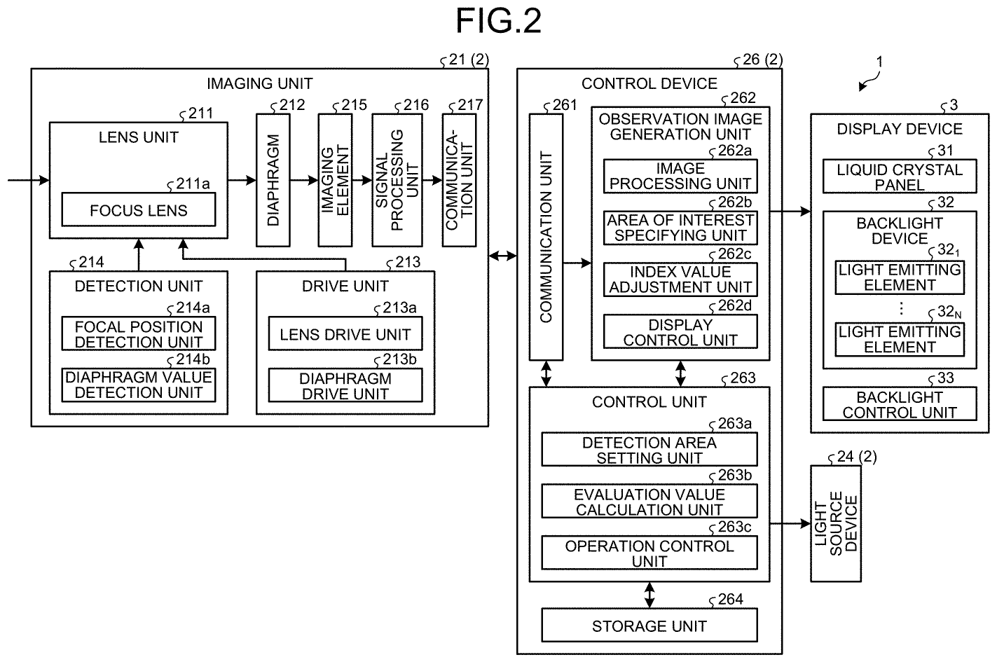

FIG. 1 is a view illustrating a medical observation system 1 according to a first embodiment. FIG. 2 is a block diagram illustrating the medical observation system 1.

The medical observation system 1 is, for example, a system that captures an image of an observation target (subject) and displays the captured image obtained by the capturing to support microsurgery such as neurosurgery or to perform endoscopic surgery. As illustrated in FIG. 1 or 2, the medical observation system 1 includes a medical observation device 2 that captures an image of an observation target, and a display device 3 that displays a captured image obtained by the capturing of the medical observation device 2.

Configuration of Medical Observation Device

The medical observation device 2 is a surgical microscope that magnifies and captures an image of a predetermined visual field area of the observation target. As illustrated in FIG. 1 or 2, the medical observation device 2 includes an imaging unit 21, a base portion 22 (FIG. 1), a support portion 23 (FIG. 1), a light source device 24, a light guide 25 (FIG. 1), and a control device 26 (FIG. 2).

As illustrated in FIG. 2, the imaging unit 21 includes a lens unit 211, a diaphragm 212, a drive unit 213, a detection unit 214, an imaging element 215, a signal processing unit 216, and a communication unit 217.

The lens unit 211 includes a focus lens 211a (FIG. 2), captures a subject image from the observation target, and forms an image on an imaging surface of the imaging element 215.

The focus lens 211a is configured with one or a plurality of lenses and adjusts a focal position by moving along an optical axis.

In addition, the lens unit 211 is provided with a focus mechanism (not illustrated) for moving the focus lens 211a along the optical axis.

The diaphragm 212 is provided between the lens unit 211 and the imaging element 215, and adjusts an amount of light of the subject image from the lens unit 211 toward the imaging element 215 under the control of the control device 26.

As illustrated in FIG. 2, the drive unit 213 includes a lens drive unit 213a and a diaphragm drive unit 213b.

In AF processing described later, which is executed by the control device 26, the lens drive unit 213a operates the above-mentioned focus mechanism under the control of the control device 26 to adjust the focal position of the lens unit 211.

The diaphragm drive unit 213b operates the diaphragm 212 under the control of the control device 26 to adjust a diaphragm value of the diaphragm 212.

As illustrated in FIG. 2, the detection unit 214 includes a focal position detection unit 214a and a diaphragm value detection unit 214b.

The focal position detection unit 214a is configured with a position sensor such as a photo interrupter, and detects a current position (focal position) of the focus lens 211a. Then, the focal position detection unit 214a outputs a signal corresponding to the detected focal position to the control device 26.

The diaphragm value detection unit 214b is configured with a linear encoder or the like, and detects a current diaphragm value of the diaphragm 212. Then, the diaphragm value detection unit 214b outputs a signal corresponding to the detected diaphragm value to the control device 26.

The imaging element 215 is configured with an image sensor that receives an image of a subject captured by the lens unit 211 and generates a captured image (analog signal).

The signal processing unit 216 performs signal processing on the captured image (analog signal) generated by the imaging element 215.

For example, the signal processing unit 216 performs processing of removing reset noise, a processing of multiplying an analog gain that amplifies the analog signal, and signal processing such as A/D conversion, for the captured image (analog signal) generated by the imaging element 215.

The communication unit 217 is an interface that communicates with the control device 26, transmits an image (digital signal) obtained by the signal processing of the signal processing unit 216 to the control device 26, and receives a control signal from the control device 26.

The base portion 22 is a base of the medical observation device 2, and is configured to be movable on a floor surface via casters 221 (FIG. 1).

The support portion 23 extends from the base portion 22 and holds the imaging unit 21 at a distal end (end portion separated from the base portion 22). Then, the support portion 23 makes the imaging unit 21 three-dimensionally movable in response to an external force applied by a manipulator.

Note that in the first embodiment, the support portion 23 is configured to have 6 degrees of freedom with respect to the movement of the imaging unit 21, but is not limited thereto, and may be configured to have a different number of other degrees of freedom.

As illustrated in FIG. 1, the support portion 23 includes first to seventh arm portions 231a to 231g, and first to sixth joint portions 232a to 232f.

The first joint portion 232a is located at the distal end of the support portion 23. The first joint portion 232a is fixedly supported by the first arm portion 231a, and holds the imaging unit 21 so as to be rotatable around a first axis O1 (FIG. 1).

Here, the first axis O1 coincides with an observation optical axis of the imaging unit 21. That is, when the imaging unit 21 is rotated around the first axis O1, a direction of the imaging field of view by the imaging unit 21 is changed.

The first arm portion 231a is a substantially rod-shaped member extending in a direction orthogonal to the first axis O1, and fixedly supports the first joint portion 232a at a distal end thereof.

The second joint portion 232b is fixedly supported by the second arm portion 231b, and holds the first arm portion 231a so as to be rotatable around a second axis O2 (FIG. 1). Therefore, the second joint portion 232b makes the imaging unit 21 rotatable around the second axis O2.

Here, the second axis O2 is orthogonal to the first axis O1 and is parallel to the extending direction of the first arm portion 231a. That is, when the imaging unit 21 is rotated around the second axis O2, a direction of the optical axis of the imaging unit 21 with respect to the observation target is changed. In other words, the field of view captured by the imaging unit 21 moves along an X axis (FIG. 1) orthogonal to the first and second axes O1 and O2 in a horizontal plane. Therefore, the second joint portion 232b is a joint portion for moving the field of view captured by the imaging unit 21 along the X axis.

The second arm portion 231b has a crank shape extending in a direction orthogonal to the first and second axes O1 and O2, and fixedly supports the second joint portion 232b at a distal end thereof.

The third joint portion 232c is fixedly supported by the third arm portion 231c, and rotatably holds the second arm portion 231b around a third axis O3 (FIG. 1). Therefore, the third joint portion 232c makes the imaging unit 21 rotatable around the third axis O3.

Here, the third axis O3 is orthogonal to the first and second axes O1 and O2. That is, when the imaging unit 21 is rotated around the third axis O3, the direction of the optical axis of the imaging unit 21 with respect to the observation target is changed. In other words, the field of view captured by the imaging unit 21 moves along a Y axis (FIG. 1) orthogonal to the X axis in the horizontal plane. Therefore, the third joint portion 232c is a joint portion for moving the field of view captured by the imaging unit 21 along the Y axis.

The third arm portion 231c is a substantially rod-shaped member extending in a direction substantially parallel to the third axis O3, and fixedly supports the third joint portion 232c at a distal end thereof.

The fourth joint portion 232d is fixedly supported by the fourth arm portion 231d, and holds the third arm portion 231c so as to be rotatable around a fourth axis O4 (FIG. 1). Therefore, the fourth joint portion 232d makes the imaging unit 21 rotatable around the fourth axis O4.

Here, the fourth axis O4 is orthogonal to the third axis O3. That is, when the imaging unit 21 is rotated around the fourth axis O4, a height of the imaging unit 21 is adjusted. Therefore, the fourth joint portion 232d is a joint portion for parallelly moving the imaging unit 21.

The fourth arm portion 231d is a substantially rod-shaped member that is orthogonal to the fourth axis O4 and linearly extends toward the base portion 22, and fixedly supports the fourth joint portion 232d on one end side.

The fifth arm portion 231e has the same shape as the fourth arm portion 231d. Then, the fifth arm portion 231e is rotatably connected to the third arm portion 231c with one end side around an axis parallel to the fourth axis O4.

The sixth arm portion 231f has substantially the same shape as the third arm portion 231c. Then, the sixth arm portion 231f is rotatably connected to the other end sides of the fourth and fifth arm portions 231d and 231e around an axis parallel to the fourth axis O4, in a posture of forming a parallelogram between the third to fifth arm portions 231c to 231e. In addition, a counterweight 233 (FIG. 1) is provided at an end portion of the sixth arm portion 231f.

The mass and arrangement position of the counterweight 233 are adjusted so that the rotational moment generated around the fourth axis O4 and the rotational moment generated around the fifth axis O5 (FIG. 1) may be offset depending on the mass of each component provided on the distal end side (the side where the imaging unit 21 is provided) of the support portion 23 with respect to the counterweight 233. That is, the support portion 23 is a balance arm (a configuration in which the counterweight 233 is provided). Note that the support portion 23 may have a configuration in which the counterweight 233 is not provided.

The fifth joint portion 232e is fixedly supported by the seventh arm portion 231g, and holds the fourth arm portion 231d so as to be rotatable around a fifth axis O5. Therefore, the fifth joint portion 232e makes the imaging unit 21 rotatable around the fifth axis O5.

Here, the fifth axis O5 is parallel to the fourth axis O4. That is, when the imaging unit 21 is rotated around the fifth axis O5, the height of the imaging unit 21 is adjusted. Therefore, the fifth joint portion 232e is a joint portion for parallelly moving the imaging unit 21.

The seventh arm portion 231g has a substantially L-shape configured with a first portion extending in a vertical direction and a second portion that bends and extends at a substantially right angle to the first portion, and fixedly supports the fifth joint portion 232e at the first portion.

The sixth joint portion 232f is fixedly supported by the base portion 22, and holds the second portion of the seventh arm portion 231g so as to be rotatable around the sixth axis O6 (FIG. 1). Therefore, the sixth joint portion 232f makes the imaging unit 21 rotatable around the sixth axis O6.

Here, the sixth axis O6 is an axis along the vertical direction. That is, the sixth joint portion 232f is a joint portion for parallelly moving the imaging unit 21.

The first axis O1 described above is configured with a passive axis that passively allows the imaging unit 21 to rotate around the first axis O1 in response to the external force applied by the manipulator, regardless of power of an actuator or the like. Similarly, the second to sixth axes O2 to O6 are also configured by passive axes.

One end of the light guide 25 is connected to the light source device 24, and illumination light of the amount of light specified by the control device 26 is supplied to one end of the light guide 25. In the first embodiment, the light source device 24 supplies white light (hereinafter, referred to as normal light) to one end of the light guide 25 as the illumination light.

One end of the light guide 25 is connected to the light source device 24, and the other end thereof is connected to the imaging unit 21. Then, the light guide 25 transmits the normal light supplied from the light source device 24 from one end to the other end and supplies the normal light to the imaging unit 21. The normal light supplied to the imaging unit 21 is irradiated to the observation target from the imaging unit 21. The normal light (subject image) that is irradiated to the observation target and reflected by the observation target is focused by the lens unit 211 in the imaging unit 21 and then captured by the imaging element 215.

The control device 26 corresponds to the medical image processing device according to the present disclosure. The control device 26 is provided inside the base portion 22 and comprehensively controls the operation of the medical observation system 1. As illustrated in FIG. 2, the control device 26 includes a communication unit 261, an observation image generation unit 262, a control unit 263, and a storage unit 264.

The communication unit 261 is an interface that communicates with the imaging unit 21 (communication unit 217), receives the captured image (digital signal) output from the imaging unit 21, and also transmits a control signal from the control unit 263.

The observation image generation unit 262 processes the captured image (digital signal) that is output from the imaging unit 21 and is received by the communication unit 261 under the control of the control unit 263. Then, the observation image generation unit 262 generates a display video signal for displaying the captured image after processing, and outputs the video signal to the display device 3. As illustrated in FIG. 2, the observation image generation unit 262 includes an image processing unit 262a, an area of interest specifying unit 262b, an index value adjustment unit 262c, and a display control unit 262d.

Note that the functions of the image processing unit 262a, the area of interest specifying unit 262b, the index value adjustment unit 262c, and the display control unit 262d will be described in “Operation of medical observation system” to be described later.

The control unit 263 is configured with, for example, a central processing unit (CPU), a field-programmable gate array (FPGA), or the like, controls the operations of the imaging unit 21, the light source device 24, and the display device 3, and controls the entire operation of the control device 26. As illustrated in FIG. 2, the control unit 263 includes a detection area setting unit 263a, an evaluation value calculation unit 263b, and an operation control unit 263c.

Note that the functions of the detection area setting unit 263a, the evaluation value calculation unit 263b, and the operation control unit 263c will be described in “Operation of medical observation system” to be described later.

The storage unit 264 stores a program executed by the control unit 263, information necessary for processing of the control unit 263, or the like.

Configuration of Display Device

As illustrated in FIG. 2, the display device 3 includes a liquid crystal panel 31, a backlight device 32, and a backlight control unit 33.

The liquid crystal panel 31 is a transmissive liquid crystal panel, and displays a captured image based on the video signal by modulating the light emitted from the backlight device 32 based on the video signal output from the observation image generation unit 262.

The backlight device 32 includes a plurality of light emitting elements 32₁ to 32_{N} such as light emitting diodes (LEDs). The plurality of light emitting elements 32₁ to 32_{N} are evenly arranged on a back side of the liquid crystal panel 31 over the entire display screen of the display device 3 (liquid crystal panel 31). Then, the plurality of light emitting elements 32₁ to 32_{N} emit light under the control of the backlight control unit 33.

The function of the backlight control unit 33 will be described in “Operation of medical observation system” to be described later.

Operation of Medical Observation System

Next, an operation of the medical observation system 1 will be described.

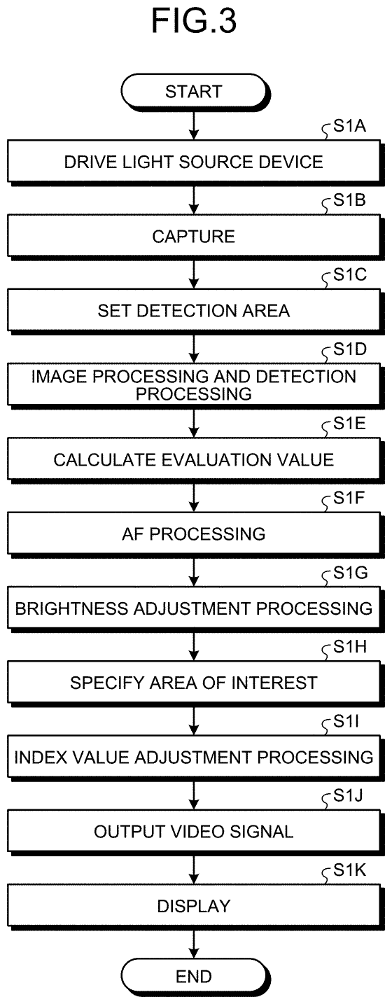

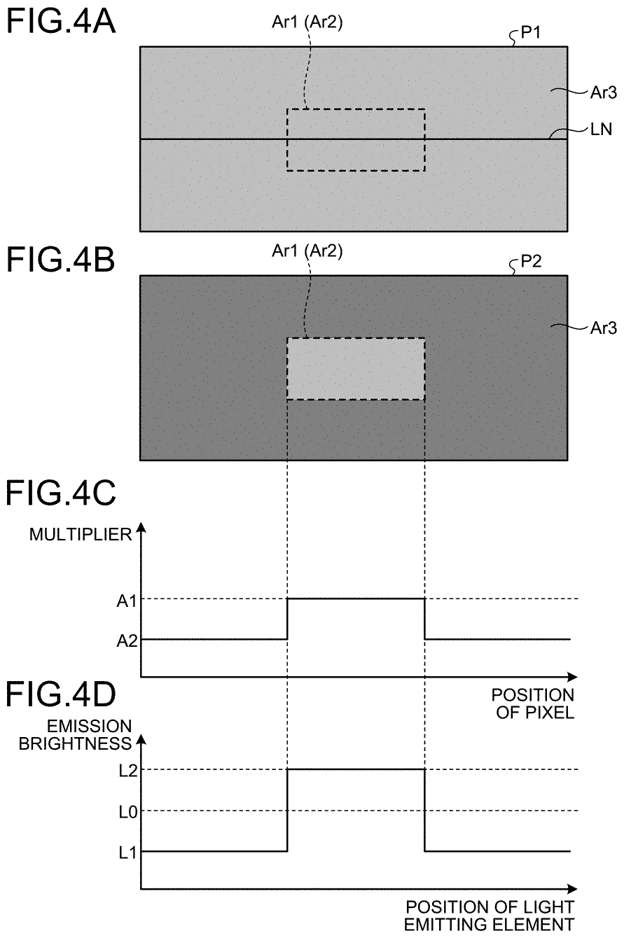

FIG. 3 is a flowchart illustrating an operation of the medical observation system 1. FIG. 4 is a diagram illustrating the operation of the medical observation system 1. Specifically, FIG. 4(a) illustrates a captured image P1 after the image processing is executed in step S1D on the captured image generated by the imaging unit 21. Note that in FIG. 4(a), for convenience of explanation, the captured image P1 has the same Y value in all the pixels. In addition, the Y value is expressed in gray scale (the Y value becomes smaller as it approaches black) in FIGS. 4(a) and 4(b). FIG. 4(b) illustrates a captured image P2 after the index value adjustment processing is executed on the captured image P1 in step S1I. FIG. 4(c) illustrates a multiplier that is multiplied by the Y value for each pixel in the captured image P1 in step S1I. In FIG. 4(c), a horizontal axis indicates a position of each pixel on one horizontal line LN in the captured image P1. A vertical axis indicates a multiplier to be multiplied for each pixel. FIG. 4(d) illustrates emission brightness of each of the light emitting element 32₁ to 32_{N} driven in step S1K. In FIG. 4(d), a horizontal axis indicates the position of each light emitting element on the horizontal line LN in the display screen of the display device 3 among the plurality of light emitting elements 32₁ to 32_{N}. A vertical axis indicates emission brightness of each light emitting element on the horizontal line LN.

First, the control unit 263 drives the light source device 24 (step S1A). As a result, the normal light emitted from the light source device 24 is irradiated from the imaging unit 21 to the observation target.

After step S1A, the control unit 263 causes the imaging element 215 to capture a subject image (normal light) that is irradiated to the observation target and reflected by the observation target at a predetermined frame rate (step S1B). Then, the imaging unit 21 captures the subject image and sequentially generates the captured image.

After step S1B, the detection area setting unit 263a sets a detection area for calculating an evaluation value used in AF processing (step S1F) and brightness adjustment processing (step S1G), which will be described later, among all the image areas in the captured image (step S1C).

Specifically, in step S1C, the detection area setting unit 263a sets a rectangular area including an image center of the captured image as the detection area among all the image areas in the captured image. Note that the detection area is not limited to the rectangular area including the center of the captured image, and may be configured so that a position of the area may be changed according to a user operation of setting the detection area to an operation input unit (not illustrated) by a manipulator such as an operator.

After step S1C, the image processing unit 262a executes the image processing and the detection processing on the captured image (digital signal) received from the imaging unit 21 via the communication unit 261 (step S1D).

Specifically, in step S1D, the image processing unit 262a executes various image processing such as digital gain processing of multiplying the captured image (digital signal) by a digital gain that amplifies the digital signal, optical black subtraction processing, white balance (WB) adjustment processing, demosaic processing, color matrix arithmetic processing, gamma correction processing, and YC conversion processing for generating a brightness signal and a color difference signal (Y, Cb/Cr signal). The captured image P1 is generated by executing the various image processing.

In addition, in step S1D, the image processing unit 262a executes the detection processing based on the captured image P1. More specifically, the image processing unit 262a executes a detection of a contrast or a frequency component of the image in the detection area Ar1, a detection of a brightness average value or the maximum and minimum pixels in the detection area Ar1 by a filter or the like, a determination of a comparison with a threshold value, and a detection of a histogram and the like, based on pixel information (e.g., Y value (brightness signal (Y signal))) for each pixel in the detection area Ar1 (FIG. 4(a)) set in step S1C among all the image areas in the captured image P1. Then, the image processing unit 262a outputs the detection information (contrast, frequency component, brightness average value, maximum and minimum pixels, histogram, and the like) obtained by the detection processing to the control unit 263.

After step S1D, the evaluation value calculation unit 263b calculates the evaluation value based on the detection information obtained by the detection processing in step S1D (step S1E).

Specifically, in step S1E, the evaluation value calculation unit 263b calculates a focusing evaluation value for evaluating a focusing state of the image in the detection area Ar1 among all the image areas in the captured image P1 based on the detection information (contrast or frequency component). For example, the evaluation value calculation unit 263b uses the contrast obtained by the detection processing in step S1D or the sum of high frequency components among the frequency components obtained by the detection processing in step S1D as the focusing evaluation value. Note that the focusing evaluation value indicates that the larger the value, the more the focus is.

In addition, in step S1E, the evaluation value calculation unit 263b calculates a brightness evaluation value for changing the brightness of the image in the detection area Ar1 among all the image areas in the captured image P1 to reference brightness (changing the detection information (brightness average value) to the reference brightness average value), based on the detection information (brightness average value). As the brightness evaluation value, first to fourth brightness evaluation values illustrated below may be exemplified.

The first brightness evaluation value is an exposure time of each pixel in the imaging element 215.

The second brightness evaluation value is an analog gain multiplied by the signal processing unit 216.

The third brightness evaluation value is a digital gain multiplied by the image processing unit 262a.

The fourth brightness evaluation value is the amount of normal light supplied by the light source device 24.

After step S1E, the operation control unit 263c executes AF processing of adjusting a focal position of the lens unit 211 (step S1F). The AF processing corresponds to a first control according to the present disclosure.

Specifically, in step S1F, the operation control unit 263c executes the AF processing for positioning the focus lens 211a at a focal position where the image in the detection area Ar1 is in focus in all the image areas of the captured image P1 by controlling the operation of the lens drive unit 213a by a hill climbing method or the like based on the focusing evaluation value calculated in step S1E and the current focal position detected by the focal position detection unit 214a.

After step S1F, the operation control unit 263c executes the brightness adjustment processing of adjusting the brightness of the image in the detection area Ar1 among all the image areas in the captured image P1 to the reference brightness (step S1G). The brightness adjustment processing corresponds to a second control according to the present disclosure.

Specifically, when the brightness evaluation value calculated in step S1E is a first brightness evaluation value, the operation control unit 263c outputs a control signal to the imaging unit 21 and uses the exposure time of each pixel of the imaging element 215 as the first brightness evaluation value. In addition, when the brightness evaluation value calculated in step S1E is a second brightness evaluation value, the operation control unit 263c outputs a control signal to the imaging unit 21 and uses the analog gain multiplied by the signal processing unit 216 as the second brightness evaluation value. Further, when the brightness evaluation value calculated in step S1E is a third brightness evaluation value, the operation control unit 263c outputs a control signal to the image processing unit 262a and uses the digital gain multiplied by the image processing unit 262a as the third brightness evaluation value. In addition, when the brightness evaluation value calculated in step S1E is a fourth brightness evaluation value, the operation control unit 263c outputs a control signal to the light source device 24 and uses the amount of normal light supplied by the light source device 24 as the fourth brightness evaluation value.

After step S1G, the area of interest specifying unit 262b specifies an area of interest Ar2 of all the image areas in the captured image P1 (step S1H).

In the first embodiment, the area of interest Ar2 is the same area as the detection area Ar1 set in step S1C, as illustrated in FIG. 4(a).

After step S1H, the index value adjustment unit 262c executes index value adjustment processing of adjusting a brightness index value, which is an index of the brightness of each pixel in the captured image P1 in order to emphasize the area of interest Ar2 with respect to the other area Ar3 in the captured image P1 (step S1I).

In the first embodiment, the brightness index value is the Y value (brightness signal (Y signal)). Then, as illustrated in FIGS. 4(b) and 4(c), the index value adjustment unit 262c multiplies the Y value for each pixel in the area of interest Ar2 in the captured image P1 by the first multiplier (constant) A1 (“1” in the first embodiment). That is, the index value adjustment unit 262c adopts the Y value as it is without adjusting the Y value for each pixel in the area of interest Ar2.

On the other hand, the index value adjustment unit 262c multiplies the Y value for each pixel in the other area Ar3 in the captured image P1 by a second multiplier (constant) A2 (e.g., “0.5”) smaller than the first multiplier A1. That is, the index value adjustment unit 262c adjusts the Y value for each pixel in the other area Ar3 so as to be darkened. By executing the index value adjustment processing, the captured image P2 in which the area of interest Ar2 is highlighted with respect to the other area Ar3 is generated.

After step S1I, the display control unit 262d generates a video signal for display for displaying the captured image P2 (luminance signal and color difference signal (Y, Cb/Cr signal)), and outputs a video signal to the display device 3 (step S1J).

After step S1J, the display device 3 displays the captured image P2 based on the video signal output from the display control unit 262d in step S1J (step S1K).

Here, in step S1K, the backlight control unit 33 controls the emission brightness of the plurality of light emitting elements 32₁ to 32_{N} by using a technique called so-called local dimming. Hereinafter, the details of the control will be described with reference to FIG. 4(d). In FIG. 4(d), the reference numeral “L0” indicates the emission brightness of each light emitting element on the horizontal line LN (hereinafter, referred to as the reference emission brightness L0) when the video signal corresponding to the captured image P1 illustrated in FIG. 4(a) is input to the display device 3.

As described above, the Y value for each pixel in the other area Ar3 is adjusted so as to be darkened. Therefore, the backlight control unit 33 controls the emission brightness of the light emitting element located in the other area Ar3 so as to be emission brightness L1 lower than the reference emission brightness L0 according to the Y value. Note that the emission brightness may be changed by controlling at least one of an applied pulse width (current supply time) and a current value of a current supplied to the light emitting element. That is, the backlight control unit 33 reduces an electric energy of the light emitting element located in the other area Ar3 from a reference electric energy to realize the reference emission brightness L0 according to the Y value.

In addition, the backlight control unit 33 uses the reduced electric energy of the light emitting element located in the other area Ar3 for the light emitting element located in the area of interest Ar2 in order to keep the electric energy of the entire backlight device 32 constant at all times. As a result, the emission brightness of the light emitting element located in the area of interest Ar2 becomes emission brightness L2 higher than the reference emission brightness L0.

As described above, the light emitted from the backlight device 32 has low brightness in the other area Ar3 while it has high brightness in the area of interest Ar2.

According to the first embodiment described above, the following effects are obtained.

The control device 26 according to the first embodiment specifies the area of interest Ar2 among all the image areas in the captured image P1. Then, the control device 26 generates the captured image P2 in which the area of interest Ar2 is highlighted with respect to the other area Ar3 by executing the index value adjustment processing.

In addition, the display device 3 emits light from the backlight device 32 to the liquid crystal panel 31 while the other area Ar3 has low brightness while the interest area Ar2 has high brightness according to the Y value for each pixel in the captured image P2.

Therefore, by both adjusting the brightness of the captured image P2 itself by the index value adjustment processing and adjusting illumination light to the liquid crystal panel 31 by the local dimming in the display device 3, the area of interest Ar2 may be further highlighted with respect to the other area Ar3. That is, the captured image P2 displayed on the display device 3 is an image suitable for observation.

Then, for example, when the display device 3 is configured with a polarized 3D image display monitor, it is possible to compensate for an attenuation of the brightness according to the transmittance of the polarized glasses worn by an observer such as an operator, and observe an image suitable for the manipulator.

By the way, for example, in the index value adjustment processing, it is conceivable to adjust the Y value for each pixel in the area of interest Ar2 so as to be bright while maintaining the Y value for each pixel in the other area Ar3 without adjusting. However, if the electric energy of the light emitting element corresponding to the Y value for each pixel in the area of interest Ar2 is close to an upper limit before the index value adjustment processing is executed, the electric energy of the light emitting element may not be increased even if the Y value is increased by the index value adjustment processing. That is, it may be difficult to emphasize the area of interest Ar2 with respect to the other area Ar3.

In the index value adjustment processing according to the first embodiment, the Y value for each pixel in the area Ar2 of interest is maintained without being adjusted, and the Y value for each pixel in the other area Ar3 is adjusted to be darkened. Therefore, the local dimming in the display device 3 may effectively generate light that makes the other area Ar3 low-brightness while the area of interest Ar2 high-brightness, and may emphasize the interest area Ar2 with respect to the other area Ar3.

By the way, since the detection area Ar1 is an area for calculating the evaluation value used for the AF processing and the brightness adjustment processing, it corresponds to an area of particular interest to the manipulator such as the operator.

Then, the control device 26 according to the first embodiment specifies the detection area Ar1 as the area of interest Ar2. Therefore, an appropriate area may be easily specified as the area of interest Ar2.

Modified Example of First Embodiment

In the first embodiment described above, the area of interest specifying unit 262b specifies the same area as the detection area Ar1 as the area of interest Ar2, but the present disclosure is not limited thereto. For example, the area of interest specifying unit 262b may simply specify the area including the image center of the captured image P1 as the area of interest without considering the detection area Ar1.

The modified example takes into consideration that the position of the imaging unit 21 may be easily adjusted so that a position where the operation is executed is located in a central area of the captured image. That is, if the area including the image center of the captured image P1 is specified as the area of interest, an appropriate area may be easily specified as the area of interest.

### Second Embodiment

Next, a second embodiment will be described.

In the following description, the same reference numerals are given to similar configurations as those of the above-described first embodiment, and a detailed description thereof will be omitted or simplified.

In the second embodiment, the index value adjustment processing (step S1I) executed by the index value adjustment unit 262c is different from that of the first embodiment described above.

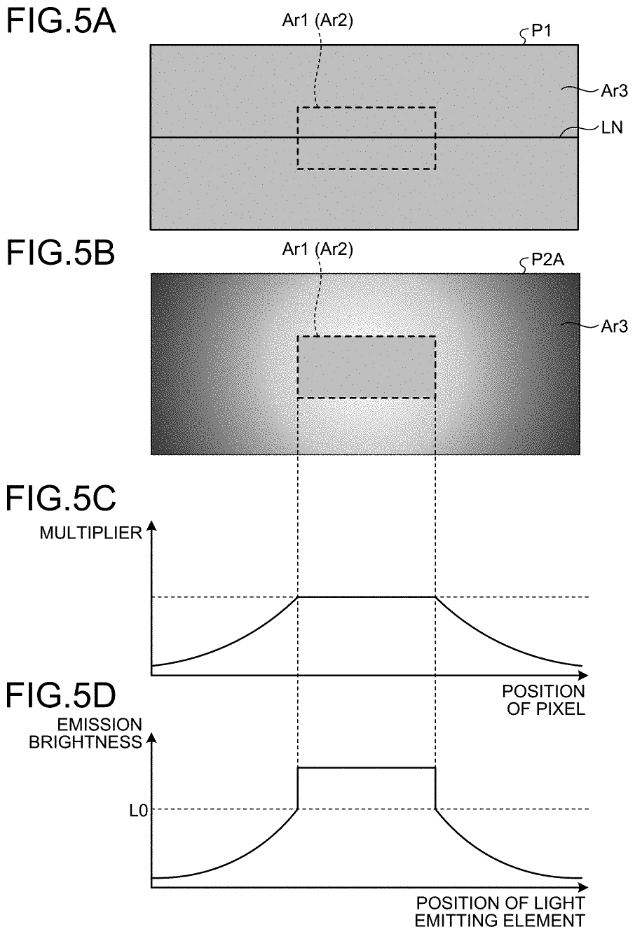

FIG. 5 is a diagram illustrating an operation of a medical observation system 1 according to the second embodiment. Specifically, FIG. 5(a) is the same diagram as FIG. 4(a). FIGS. 5(b) to 5(d) are diagrams corresponding to FIGS. 4(b) to 4(d), respectively.

In the second embodiment, as illustrated in FIGS. 5(b) and 5(c), the index value adjustment unit 262c multiplies the Y value for each pixel of the area of interest Ar2 in the captured image P1 by the first multiplier (constant) A1 (“1” in the second embodiment). That is, the index value adjustment unit 262c adopts the Y value as it is without adjusting the Y value for each pixel in the area of interest Ar2, similarly to the first embodiment.

On the other hand, the index value adjustment unit 262c multiplies the Y value for each pixel of the other area Ar3 in the captured image P1 by a multiplier that becomes smaller than the first multiplier as a distance from the area of interest Ar2 increases. By executing the index value adjustment processing, a captured image P2A (FIG. 5(b)) in which the area of interest Ar2 is highlighted with respect to the other area Ar3 is generated.

In addition, the details of the control of the emission brightness of the plurality of light emitting elements 32₁ to 32_{N} by the backlight control unit 33 will be described with reference to FIG. 5(d).

As described above, the Y value for each pixel in the other area Ar3 is adjusted so as to become smaller as the distance from the area of interest Ar2 increases. Therefore, the backlight control unit 33 controls the emission brightness of the light emitting element located in the other area Ar3 so as to be lower than the reference emission brightness L0 as the distance from the area of interest Ar2 increases according to the Y value.

In addition, the backlight control unit 33 uses the reduced electric energy of the light emitting element located in the other area Ar3 for the light emitting element located in the area of interest Ar2 in order to keep the electric energy of the entire backlight device 32 constant at all times. As a result, the emission brightness of the light emitting element located in the area of interest Ar2 becomes higher than the reference emission brightness L0.

As described above, the light emitted from the backlight device 32 becomes low-brightness as the other area Ar3 move outward, while the light in the area of interest Ar2 becomes high-brightness.

Even when the index value adjustment processing is executed as in the second embodiment described above, the same effect as that of the first embodiment described above is obtained.

### Third Embodiment

Next, a third embodiment will be described.

In the following description, the same reference numerals are given to similar configurations as those of the above-described first embodiment, and a detailed description thereof will be omitted or simplified.

In the third embodiment, the index value adjustment process (step S1I) executed by the index value adjustment unit 262c is different from that of the first embodiment described above.

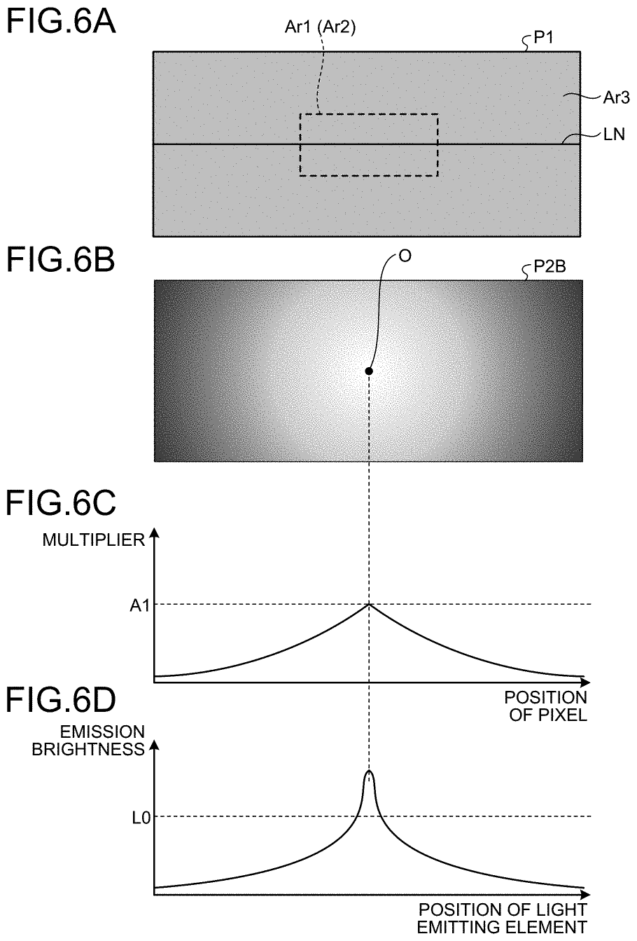

FIGS. 6(a) to 6(b) are diagrams illustrating an operation of a medical observation system 1 according to a third embodiment. Specifically, FIG. 6(a) is the same diagram as FIG. 4(a). FIGS. 6(b) to 6(d) are diagrams corresponding to FIGS. 4(b) to 4(d), respectively.

In the third embodiment, as illustrated in FIGS. 6(b) and 6(d), the index value adjustment unit 262c multiplies the Y value of each pixel in all the image areas of the captured image P1 by a multiplier that decreases as a distance from the center position O of the area of interest Ar2 increases. Note that the multiplier to be multiplied by the Y value of the pixel at the center position O of the area of interest Ar2 is a first multiplier A1 (“1” in the third embodiment). By executing the index value adjustment processing, a captured image P2B (FIG. 6(b)) which becomes darker as the distance from the center position of the area of interest Ar2 increases, in other words, in which the area of interest Ar2 is highlighted with respect to the other area Ar3 is generated.

In addition, the details of the control of the emission brightness of the plurality of light emitting elements 32₁ to 32_{N} by the backlight control unit 33 will be described with reference to FIG. 6(d).

As described above, the Y value for each pixel in all the image areas in the captured image P1 is adjusted so as to become smaller as the distance from the center position O of the area of interest Ar2 increases. Therefore, the backlight control unit 33 controls the emission brightness of the light emitting elements other than the light emitting element located near the center position O of the area of interest Ar2 among the plurality of light emitting elements 32₁ to 32_{N} so as to be lower than the reference emission brightness L0 as the distance from the center position O increases according to the Y value.

In addition, the backlight control unit 33 uses the reduced electric energy for the light emitting element other than the light emitting element located near the center position O of the area of interest Ar2 for the light emitting element located near the center position O in order to keep the electric energy of the entire backlight device 32 constant at all times. As a result, emission brightness of the light emitting element located near the center position O becomes higher than the reference emission brightness L0.

As described above, the light emitted from the backlight device 32 has high brightness near the center of the area of interest Ar2 and low brightness toward the outside from the center.

According to the third embodiment described above, in addition to the same effect as that of the first embodiment described above, the following effect is obtained.

In the captured image P2B displayed on the display device 3, since a boundary between the area of interest Ar2 and the other area Ar3 disappears, the image will not be uncomfortable for the manipulator such as the operator who observes the captured image P2B.

### Fourth Embodiment

Next, a fourth embodiment will be described.

In the following description, the same reference numerals are given to similar configurations as those of the above-described first embodiment, and a detailed description thereof will be omitted or simplified.

In the fourth embodiment, a function of executing enlargement processing is added to the image processing unit 262a and the index value adjustment processing (step S1I) executed by the index value adjustment unit 262c are different from the first embodiment described above.

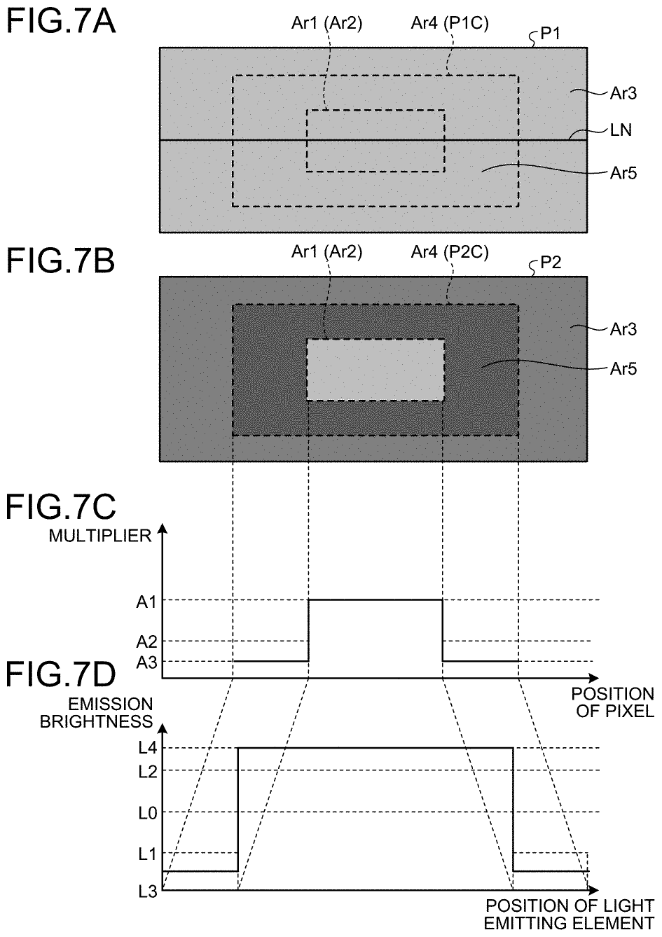

FIG. 7 is a diagram illustrating an operation of a medical observation system 1 according to the fourth embodiment. Specifically, FIG. 7(a) is the same diagram as FIG. 4(a). FIGS. 7(b) to 7(d) are diagrams corresponding to FIGS. 4(b) to 4(d), respectively. Note that FIGS. 7(c) and 7(d) illustrate a multiplier and emission brightness according to a captured image P1C after the enlargement processing, respectively.

In the fourth embodiment, the image processing unit 262a executes the enlargement processing according to a user operation for executing the enlargement processing to the operation input unit (not illustrated) by the manipulator such as the operator. Specifically, the image processing unit 262a cuts out a specific area Ar4 including the area of interest Ar2 in the captured image P1. Then, the image processing unit 262a enlarges the area Ar4 to generate the captured image P1C in order to display the area Ar4 in the captured image P1 on the entire display screen of the display device 3. That is, the image processing unit 262a corresponds to the enlargement processing unit according to the present disclosure.

Hereinafter, the operations of the index value adjustment unit 262c and the backlight control unit 33 after the enlargement processing described above is executed will be described. Note that before the enlargement processing is executed, the index value adjustment unit 262c and the backlight control unit 33 execute the same operations as the operations (steps S1I and S1K) described in the first embodiment described above.

In the fourth embodiment, as illustrated in FIGS. 7(b) and 7(c), the index value adjustment unit 262c multiplies the Y value for each pixel of the area of interest Ar2 in the captured image P1C by the same first multiplier (constant) A1 (“1” in the fourth embodiment) as before the enlargement processing is executed, after the enlargement processing is executed. That is, there is no change in the Y value for each pixel of the area of interest Ar2 before and after the enlargement processing is executed.

On the other hand, the index value adjustment unit 262c multiplies a Y value for each pixel of an area Ar5 other than the area of interest Ar2 in the captured image P1C by a third multiplier (constant) A3 (e.g., “0.25”) that is smaller than the second multiplier (constant) A2 (e.g., “0.5”) that is multiplied before the enlargement processing is executed. That is, when the enlargement processing is executed, the other area Ar5 becomes dark. By executing the index value adjustment processing, a captured image P2C (FIG. 7(b)) in which the area of interest Ar2 is highlighted with respect to the other area Ar5 is generated.

In addition, the details of the control of the emission brightness of the plurality of light emitting elements 32₁ to 32_{N} by the backlight control unit 33 will be described with reference to FIG. 7(d).

As described above, the Y value of the pixel value in the other area Ar5 is adjusted so as to become darker than before the enlargement processing is executed when the enlargement processing is executed. Therefore, the backlight control unit 33 controls the emission brightness of the light emitting element located in the other area Ar5 so as to be emission brightness L3 lower than the emission brightness L1 before the enlargement processing is executed, according to the Y value.

In addition, the backlight control unit 33 uses the reduced electric energy of the light emitting element located in the other area Ar5 for the light emitting element located in the area of interest Ar2 in order to keep the electric energy of the entire backlight device 32 constant at all times. As a result, the emission brightness of the light emitting element located in the area of interest Ar2 becomes emission brightness L4 higher than the emission brightness L2 before the enlargement processing is executed.

As described above, the light emitted from the backlight device 32 has low brightness in the other area Ar5 while it has high brightness in the area of interest Ar2.

According to the fourth embodiment described above, in addition to the same effect as that of the first embodiment described above, the following effects are obtained.

Here, a ratio of the other area Ar3 occupied in the captured image P2 before the enlargement processing is executed is set as a first ratio. In addition, a ratio of the other area Ar5 occupied in the captured image P2C after the enlargement processing is executed is set as a second ratio. Then, the second ratio is smaller than the first ratio. Therefore, if the multiplier that is multiplied on the other area Ar3 before the enlargement processing is executed and the multiplier that is multiplied on the other area Ar5 after the enlargement processing is executed are the same, the following phenomena will occur.

That is, since the second ratio is smaller than the first ratio, the electric energy to be reduced from the reference electric energy (the electric energy to realize the reference emission brightness L0) for the light emitting element located in the other area Ar5 is also smaller than that before the enlargement processing is executed. Therefore, when the reduced electric energy of the light emitting element located in the other area Ar5 is used for the light emitting element located in the area of interest Ar2, the brightness of the area of interest Ar2 may be lower than before the enlargement processing was executed.

In the fourth embodiment, the multiplier to be multiplied to the other area Ar5 after the enlargement processing is executed is smaller than the multiplier that is multiplied to the other area Ar3 before the enlargement processing is executed. Therefore, the phenomenon described above does not occur, and an image suitable for observation may be generated even when the enlargement processing is executed.

### Fifth Embodiment

Next, a fifth embodiment will be described.

In the following description, the same reference numerals are given to similar configurations as those of the above-described first embodiment, and a detailed description thereof will be omitted or simplified.

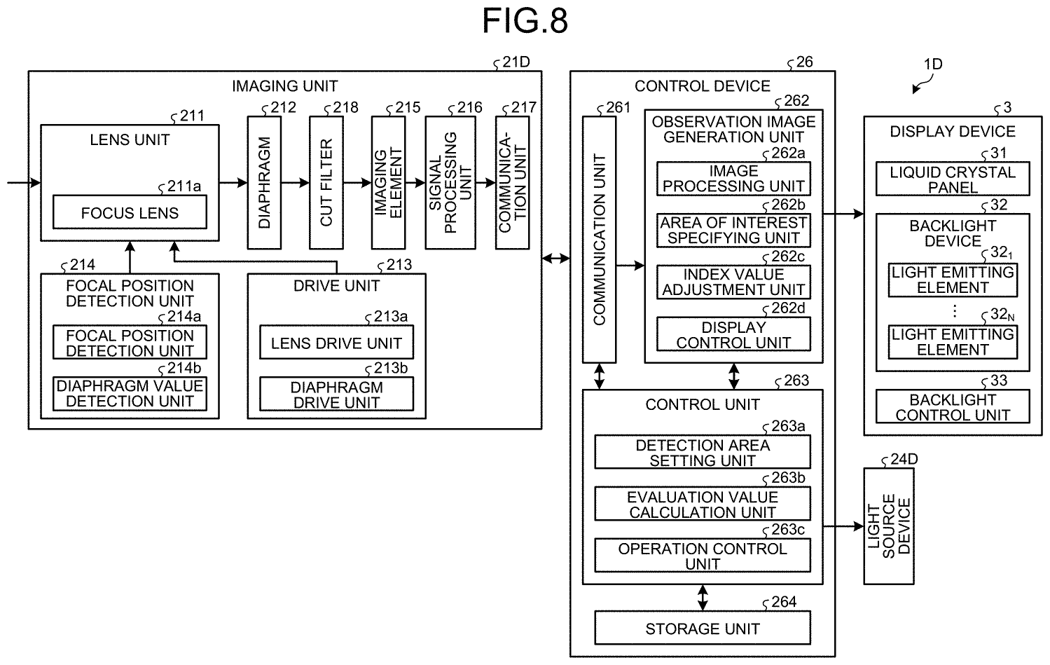

FIG. 8 is a block diagram illustrating a medical observation system 1D according to a fifth embodiment.

The medical observation system 1D according to the fifth embodiment is a system for performing photodynamic diagnosis, which is one of cancer diagnostic methods for detecting cancer cells.

Specifically, in the photodynamic diagnosis, for example, a photosensitive substance such as 5-aminolevulinic acid (hereinafter, referred to as 5-ALA) is used. The 5-ALA is a natural amino acid originally contained in a living body of animals and plants. This 5-ALA is taken up into cells after administration into the body and biosynthesized into protoporphyrin in mitochondria. Then, in the cancer cells, the protoporphyrin is excessively accumulated. In addition, the protoporphyrin that is excessively accumulated in the cancer cells has photoactivity. Therefore, when the protoporphyrin is excited with excitation light (e.g., blue visible light in a wavelength band of 375 nm to 445 nm), it emits fluorescence (e.g., red fluorescence in a wavelength band of 600 nm to 740 nm). In this way, the cancer cell method in which the photosensitive substance is used to fluoresce the cancer cells is called photodynamic diagnosis.

Then, in the medical observation system 1D according to the fifth embodiment, as illustrated in FIG. 8, the configuration of the light source device 24 and the imaging unit 21 is changed with respect to the medical observation system 1 described in the first embodiment described above. Hereinafter, for convenience of explanation, the light source device 24 and the imaging unit 21 according to the fifth embodiment will be referred to as a light source device 24D and an imaging unit 21D, respectively.

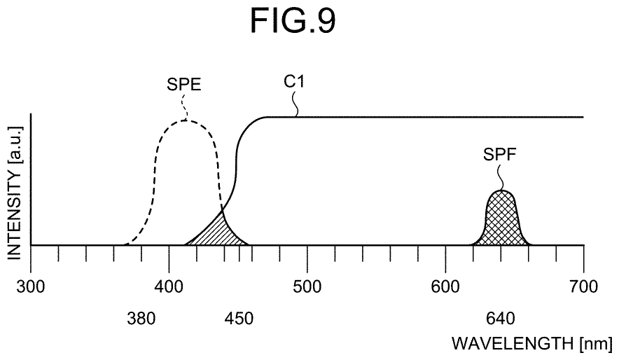

FIG. 9 is a diagram illustrating a spectrum of light emitted from a light source device 24D.

The light source device 24D has a different emission light from the light source device 24 described in the first embodiment described above. Specifically, the light source device 24D is configured with an LED, a semiconductor laser, or the like, and emits excitation light. In the fifth embodiment, the excitation light is excitation light in a blue wavelength band (e.g., a wavelength band of 375 nm to 445 nm) that excites protoporphyrin, as in a spectral SPE illustrated in FIG. 9. In addition, the protoporphyrin emits fluorescence in a red wavelength band (e.g., a wavelength band of 600 nm to 740 nm) when excited by the excitation light, as in the spectral SPF illustrated in FIG. 9. Then, the excitation light emitted from the light source device 24D and supplied to the imaging unit 21D via the light guide 25 is irradiated from the imaging unit 21D to the observation target. The excitation light irradiated to the observation target and reflected by the observation target and the fluorescence excited by the protoporphyrin accumulated in a lesion portion of the observation target and emitted from the protoporphyrin are focused by the lens unit 211 in the imaging unit 21D, and then captured by the imaging element 215.

In the imaging unit 21D, a cut filter 218 is added to the imaging unit 21 described in the first embodiment described above.

The cut filter 218 is provided between the diaphragm 212 and the imaging element 215, and has a transmission characteristic of transmitting light in a wavelength band of about 410 nm or more, as illustrated by a curve C1 in FIG. 9. That is, the cut filter 218 transmits all of the subject images (excitation light and fluorescence) from the diaphragm 212 to the imaging element 215 for fluorescence and transmits only a part of the excitation light.

Next, an operation of the medical observation system 1D will be described.

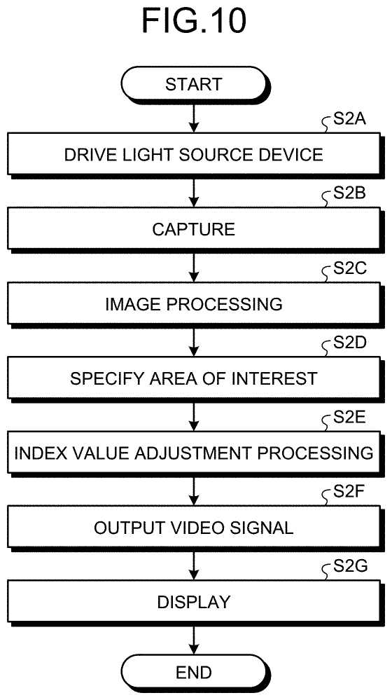

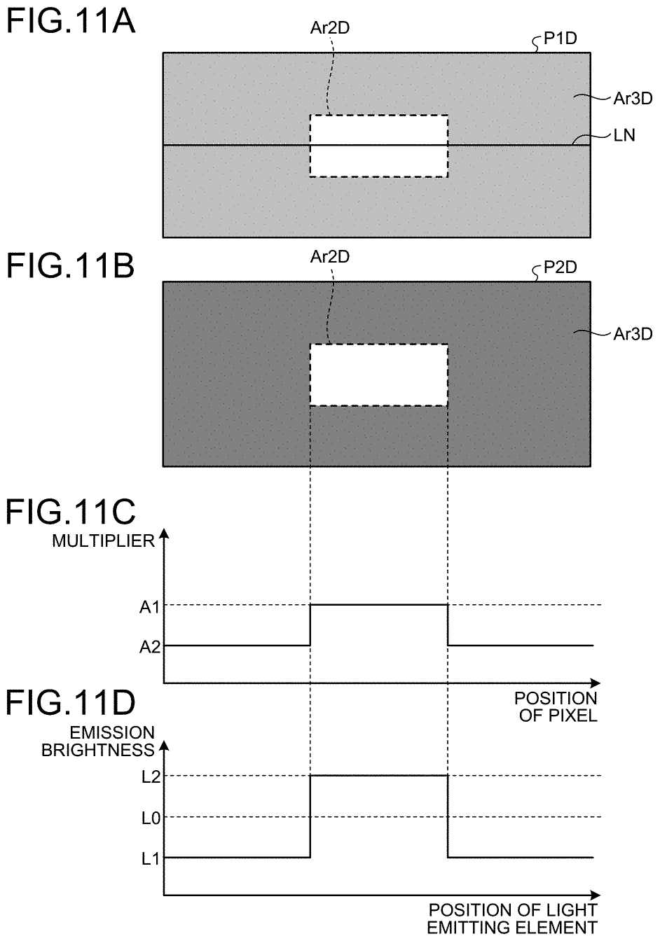

FIG. 10 is a flowchart illustrating the operation of the medical observation system 1D. FIG. 11 is a diagram illustrating the operation of the medical observation system 1D. Specifically, FIG. 11(a) illustrates a captured image P1D after the image processing is executed in step S2C on the captured image generated by the imaging unit 21D. In FIGS. 11(a) and 11(b), an area (fluorescent area Ar2D) in which the protoporphyrin excited by the excitation light fluoresces is represented in white. In addition, it is assumed that the Y value is the same for the fluorescent area Ar2D. Further, it is assumed that an area Ar3D other than the fluorescence area Ar2D has a constant Y value different from that of the fluorescence area Ar2D, and the Y value is represented in gray scale (the Y value becomes smaller as it approaches black). FIG. 11(b) illustrates a captured image P2D after the index value adjustment processing is executed on the captured image P1D in step S2E. FIG. 11(c) is a diagram corresponding to FIG. 4(c), and illustrates a multiplier to be multiplied by the Y value for each pixel in the captured image P1D in step S2E. FIG. 11(d) is a diagram corresponding to FIG. 4(d), and illustrates the emission brightness of each of the light emitting element 32₁ to 32_{N} driven in step S2G.

First, the control unit 263 drives the light source device 24D (step S2A). As a result, the excitation light emitted from the light source device 24D is irradiated from the imaging unit 21 to the observation target.

After step S2A, the control unit 263 causes the imaging element 215 to capture the subject image (excitation light and fluorescence) at a predetermined frame rate (step S2B). Then, the imaging unit 21D captures the subject image and sequentially generates the captured image.

After step S2B, the image processing unit 262a executes the same image processing as step S1D described in the first embodiment described above on the captured image (digital signal) received from the imaging unit 21D via the communication unit 261 (step S2C). The captured image P1D is generated by executing the image processing on the captured image generated by the imaging unit 21D.

After step S2C, the area of interest specifying unit 262b specifies an area of interest among all the image areas in the captured image P1D (step S2D).

Specifically, in step S2D, the area of interest specifying unit 262b specifies a fluorescence area Ar2D in which the intensity of a fluorescence component is a specific threshold value or more among all the image areas in the captured image P1D as the area of interest. Here, as the intensity of the fluorescent component, a Y value or an R value in a pixel value (RGB value) in which the fluorescent component mainly appears may be exemplified. That is, the area of interest specifying unit 262b specifies an area in which the Y value or the R value is the specific threshold value or more as the area of interest.

After step S2D, the medical observation system 1D executes steps S2E to S2G similar to steps S1I to S1K described in the first embodiment described above. In steps S2E to S2G, it is only different that the captured images P1 and P2, the area of interest Ar2, and the other area Ar3 are set as the captured images P1D and P2D, the area of interest Ar2D, and the other area Ar3E with respect to the steps S1I to S1K.

Even when the present disclosure is applied to the medical observation system 1D for performing the photodynamic diagnosis as in the fifth embodiment described above, the same effect as that of the first embodiment described above is obtained.

### Sixth Embodiment

Next, a sixth embodiment will be described.

In the following description, the same reference numerals are given to similar configurations as those of the above-described first embodiment, and a detailed description thereof will be omitted or simplified.

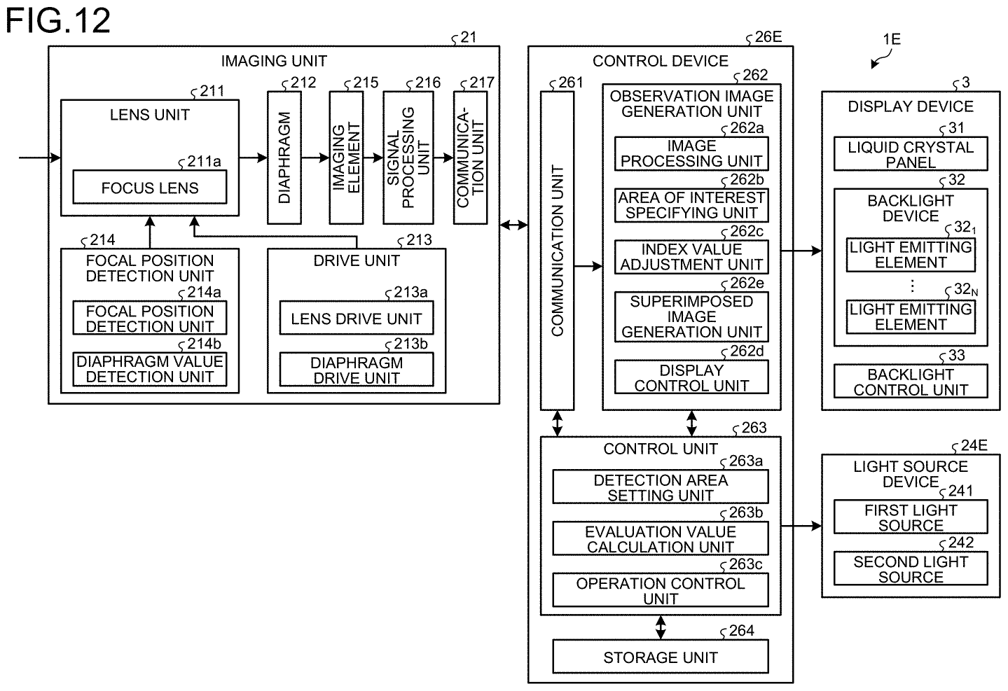

FIG. 12 is a block diagram illustrating a medical observation system 1E according to a sixth embodiment.

The medical observation system 1E according to the sixth embodiment is a system for performing ICG fluorescence observation in which indocyanine green is administered into an observation target and fluorescence from the indocyanine green excited by excitation light is observed.

Then, in the medical observation system 1E according to the sixth embodiment, as illustrated in FIG. 12, the configuration of the light source device 24 and the control device 26E is changed with respect to the medical observation system 1 described in the first embodiment described above. Hereinafter, for convenience of explanation, the light source device 24 and the control device 26 according to the sixth embodiment will be referred to as a light source device 24E and a control device 26E, respectively.

The light source device 24E emits light differently from the light source device 24 described in the first embodiment described above. Specifically, the light source device 24E includes a first light source 241 and a second light source 242, as illustrated in FIG. 12.

The first light source 241 is configured with an LED, a semiconductor laser, or the like, and emits light in a first wavelength band. In the sixth embodiment, the first light source 241 emits white light (hereinafter, referred to as normal light) as the light in the first wavelength band. Then, the normal light emitted from the first light source 241 and supplied to the imaging unit 21 via the light guide 25 is irradiated from the imaging unit 21 to the observation target. The normal light that is irradiated to the observation target and reflected by the observation target is focused by the lens unit 211 in the imaging unit 21 and then captured by the imaging element 215. Note that in the following, the normal light focused by the lens unit 211 will be referred to as a first subject image. In addition, a captured image generated by the imaging element 215 by capturing the first subject image is referred to as a normal light image.

The second light source 242 is configured with an LED, a semiconductor laser, or the like, and emits near-infrared excitation light in a near-infrared wavelength band that excites indocyanine green. Then, the near-infrared excitation light emitted from the second light source 242 and supplied to the imaging unit 21 via the light guide 25 is irradiated from the imaging unit 21 to the observation target. The near-infrared excitation light that is irradiated to the observation target and reflected by the observation target and the fluorescence excited by the indocyanine green in the observation target and emitted from the indocyanine green are focused by the lens unit 211 in the imaging unit 21, and then captured by the imaging element 215. In the following, the near-infrared excitation light and fluorescence focused by the lens unit 211 will be referred to as a second subject image. In addition, a captured image generated by the imaging element 215 by capturing the second subject image is referred to as a fluorescence image.

In the control device 26E, a superimposed image generation unit 262e is added to the observation image generation unit 262 with respect to the control device 26 described in the first embodiment described above.

The function of the superimposed image generation unit 262e will be described when an operation of a medical observation system 1E described later is described.

Next, an operation of a medical observation system 1E will be described.

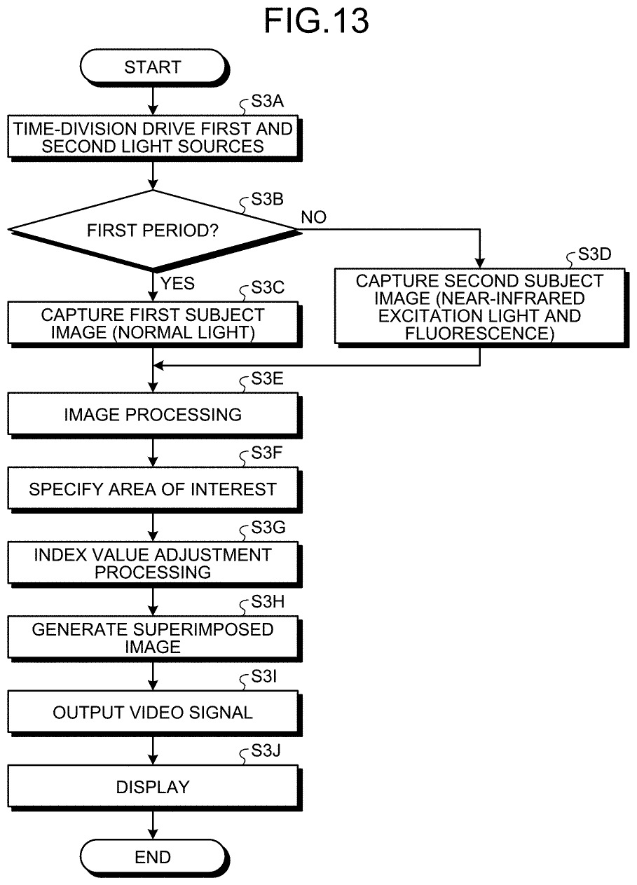

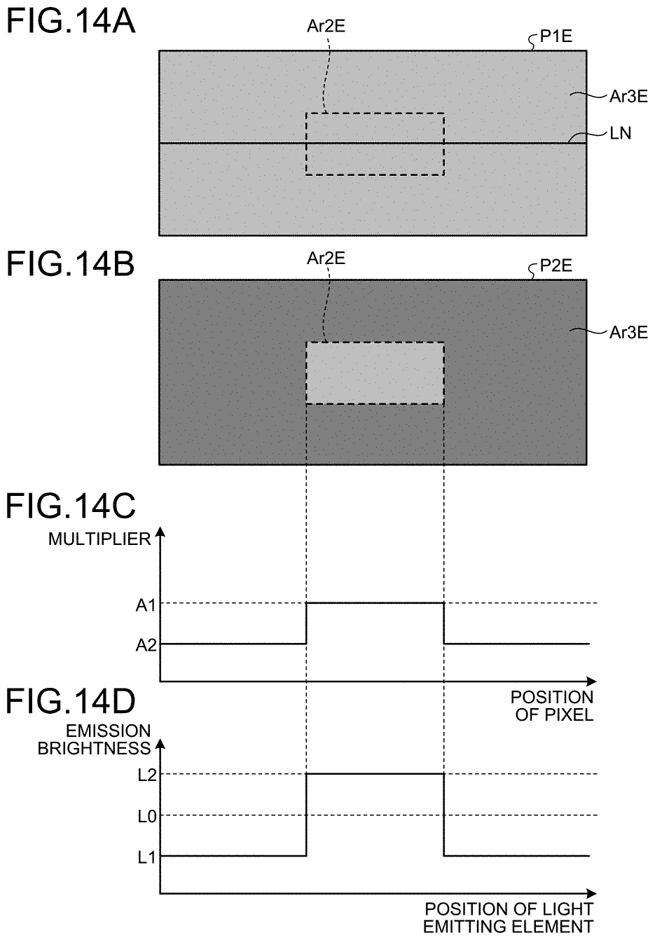

FIG. 13 is a flowchart illustrating the operation of the medical observation system 1E. FIG. 14 is a diagram illustrating the operation of the medical observation system 1E. Specifically, FIG. 14(a) illustrates a normal light image P1E after the image processing is executed in step S3E on the normal light image generated by the imaging unit 21. In FIG. 14(a), for convenience of explanation, the normal light image P1E has the same Y value in all the pixels. In addition, the Y value is expressed in gray scale (the Y value becomes smaller as it approaches black) in FIGS. 14(a) and 14(b). FIG. 14(b) illustrates a normal light image P2E after the index value adjustment processing is executed on the normal light image P1E in step S3G. FIG. 14(c) is a diagram corresponding to FIG. 4(c), and illustrates a multiplier to be multiplied by the Y value for each pixel in the normal light image P1E in step S3G. FIG. 14(d) is a diagram corresponding to FIG. 4(d), and illustrates the emission brightness of each of the light emitting element 32₁ to 32_{N} driven in step S3J.

First, the control unit 263 executes time-division driving of the first and second light sources 241 and 242 (step S3A). Specifically, in step S3A, the control unit 263 emits the normal light from the first light source 241 in a first period of the first and second periods that are alternately repeated based on a synchronization signal and emits the near-infrared excitation light from the second light source 242 in the second period.

After step S3A, the control unit 263 synchronizes with light emission timings of the first and second light sources 241 and 242 based on the synchronization signal, and causes the imaging element 215 to capture the first and second subject images in the first and second periods (steps S3B to S3D). That is, when the imaging element 215 is in the first period (step S3B: Yes), in other words, when the observation target is irradiated with the normal light, the imaging element 215 captures the first subject image (normal light) to generate a normal light image (step S3C). On the other hand, when the imaging element 215 is in the second period (step S3B: No), in other words, when the observation target is irradiated with the near-infrared excitation light, the imaging element 215 captures the second subject image (near-infrared excitation light and fluorescence) to generate a fluorescence image (step S3D).

After step S3C and step S3D, the image processing unit 262a executes the same image processing as step S1D described in the first embodiment described above on the normal light image (digital signal) and the fluorescent image (digital signal) received from the imaging unit 21 via the communication unit 261 (step S3E). The normal light image P1E is generated by executing the image processing on the normal light image generated by the imaging unit 21.

After step S3E, the area of interest specifying unit 262b specifies an area of interest of all the image areas in the normal light image P1E (step S3F).

Specifically, in step S3F, the area of interest specifying unit 262b specifies a fluorescence area in which the intensity of a fluorescence component is a specific threshold value or more among all the image areas in the fluorescence image after the image processing is executed in step S3E. Here, as the intensity of the fluorescent component, a Y value or an R value in a pixel value (RGB value) in which the fluorescent component mainly appears may be exemplified. Then, the area of interest specifying unit 262b specifies an area corresponding to the fluorescence area as an area of interest Ar2E in the normal light image P1E.

After step S3F, the index value adjustment unit 262c executes step S3G similar to step S1I described in the first embodiment described above. In step S3G, it is only different that the captured images P1 and P2, the area of interest Ar2, and the other area Ar3 are set as the normal light images P1E and P2E, the area of interest Ar2E, and the other area Ar3E with respect to the step S1I.

After step S3G, the superimposed image generation unit 262e executes superimposition processing of superimposing the fluorescent image after the image processing is executed in step S3E on the normal light image P2E to generate a superimposed image (step S3H).

Here, as the superimposition processing, first superimposition processing and second superimposition processing illustrated below may be exemplified.

The first superimposition processing is processing of replacing the area of interest Ar2E with an image of the fluorescence area in the fluorescence image in the normal light image P2E.

The second superimposition processing is processing of changing brightness of a color indicating fluorescence attached to each pixel of the area of interest Ar2E in the normal light image P2E according to a brightness value of each pixel position in the fluorescence area of the fluorescent image.

After step S3H, the medical observation system 1E executes steps S3I and S3J similar to steps S1J and S1K described in the first embodiment described above. In steps S3I and S3J, it is only different that the captured image P2, the area of interest Ar2, and the other area Ar3 are the superimposed image, the area of interest Ar2E, and the other area Ar3E with respect to the steps S1J and S1K.

Even when the present disclosure is applied to the medical observation system 1E for performing the ICG fluorescence observation as in the sixth embodiment described above, the same effect as that of the first embodiment described above is obtained.

In the sixth embodiment described above, the Y value for each pixel in all the image areas of the normal light image P1E may be adjusted to be darkened.

### Seventh Embodiment

Next, a seventh embodiment will be described.

In the following description, the same reference numerals are given to similar configurations as those of the above-described first embodiment, and a detailed description thereof will be omitted or simplified.

In the first embodiment described above, the present disclosure has been applied to the medical observation system 1 using the surgical microscope (medical observation device 2).

On the other hand, in the seventh embodiment, the present disclosure is applied to a medical observation system using a rigid endoscope.

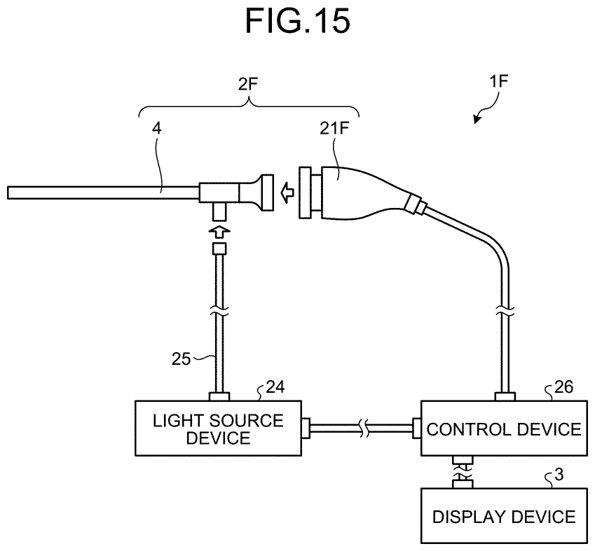

FIG. 15 is a view illustrating a medical observation system 1F according to a seventh embodiment.

As illustrated in FIG. 15, the medical observation system 1F according to a seventh embodiment includes a rigid endoscope 2F, the light source device 24 that is connected to the rigid endoscope 2F via the light guide 25 and generates illumination light emitted from the distal end of the rigid endoscope 2F, the control device 26 that processes the captured image output from the rigid endoscope 2F, and the display device 3 that displays the captured image based on the video signal for display processed by the control device 26.

As illustrated in FIG. 15, the rigid endoscope 2F includes an insertion portion 4 and a camera head 21F.

The insertion portion 4 has an elongated shape in which the whole is hard, or a part is soft and the other part is hard, and is inserted into the living body. Then, the insertion portion 4 captures light (subject image) from the living body (subject).

The camera head 21F is detachably connected to a proximal end (eyepiece portion) of the insertion portion 4. The camera head 21F has substantially the same configuration as the imaging unit 21 described in the first embodiment described above. Then, the camera head 21F captures the subject image captured by the insertion portion 4 and outputs the captured image.

Even when the rigid endoscope 2F is used as in the seventh embodiment described above, the same effect as that of the first embodiment described above is obtained.

### Eighth Embodiment

Next, an eighth embodiment will be described.

In the following description, the same reference numerals are given to similar configurations as those of the above-described first embodiment, and a detailed description thereof will be omitted or simplified.

In the first embodiment described above, the present disclosure has been applied to the medical observation system 1 using the surgical microscope (medical observation device 2).

On the other hand, in the eighth embodiment, the present disclosure is applied to a medical observation system 1 using a flexible endoscope.

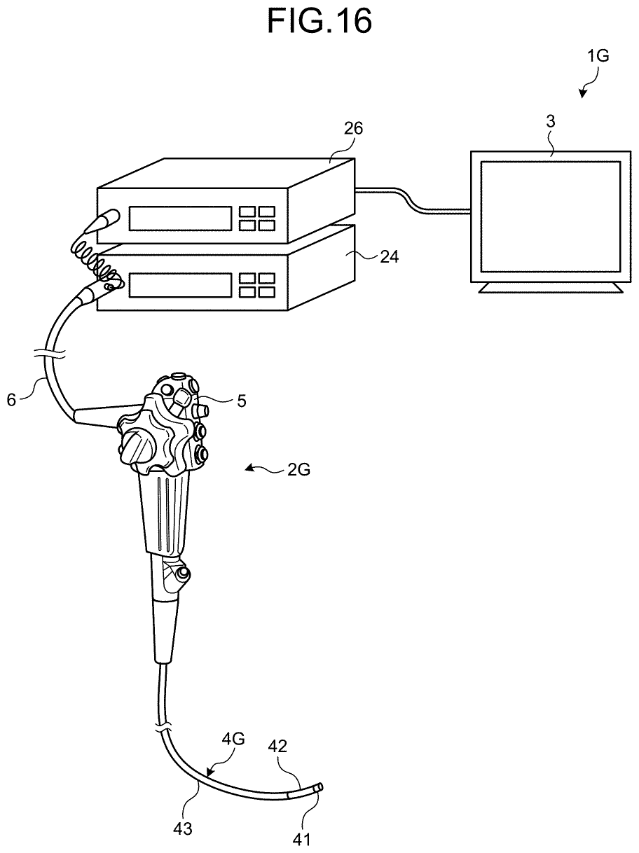

FIG. 16 is a view illustrating a medical observation system 1G according to an eighth embodiment.

As illustrated in FIG. 16, the medical observation system 1G according to the eighth embodiment includes a flexible endoscope 2G that captures an in-vivo image of an observed region by inserting an insertion portion 4G into a living body and outputs a captured image, the light source device 24 that generates the illumination light emitted from a distal end of the flexible endoscope 2G, the control device 26 that processes the captured image output from the flexible endoscope 2G, and the display device 3 that displays the captured image based on the video signal for display processed by the control device 26.

As illustrated in FIG. 16, the flexible endoscope 2G includes a flexible and elongated insertion portion 4G, an operating portion 5 connected to a proximal end side of the insertion portion 4G and accepting various operations, and a universal cord 6 that extends from the operating portion 5 in a direction different from a direction in which the insertion portion 4G extends and contains various cables connected to the light source device 24 and the control device 26.

As illustrated in FIG. 16, the insertion portion 4G includes a distal end portion 41, a bendable bending portion 42 connected to a proximal end side of the distal end portion 41 and configured with a plurality of bending pieces, and a flexible long flexible tube portion 43 connected to a proximal end side of the bending portion 42 and having flexibility.

Although a specific illustration is omitted, a configuration substantially similar to that of the imaging unit 21 described in the first embodiment described above is built in the distal end portion 41. Then, the captured image from the distal end portion 41 is output to the control device 26 via the operating portion 5 and the universal cord 6.

Even when the flexible endoscope 2G is used as in the eighth embodiment described above, the same effect as that of the first embodiment described above is obtained.

### Other Embodiments

The embodiments for carrying out the present disclosure have been described above, but the present disclosure should not be limited only to the first to eighth embodiments described above.

In the first to eighth embodiments described above, brightness of an area other than the area of interest may be adjusted to make the brightness of the area of interest constant when the captured image is a moving image. For example, when the moving image is displayed on a monitor display, it may be hard for an observer to see the image if each frame of the area of interest has different brightness. Moreover, it may be hard to see the image if each frame of the area of interest has different brightness when switching between a normal light image observation and a special light observation. Therefore, the emission brightness of the light emitting element may be controlled by adjusting the index value of the area other than the area of interest such that the brightness of the area of interest is maintained constant for a predetermined period (for example, during the capturing the observation target, displaying the captured moving image of the observation target or reproducing the moving image) for a plurality of frames.

In the first to eighth embodiments described above, the Y value (brightness signal (Y signal)) is adopted as the brightness index value according to the present disclosure, but the present disclosure is not limited thereto. For example, a Cb value, a Cr value, or a pixel value (RGB value) may be adopted as the brightness index value according to the present disclosure.

In the medical observation device 2 according to the first to sixth embodiments described above, the first to sixth axes O1 to O6 are respectively configured with passive axes, but are not limited thereto. At least one of the first to sixth axes O1 to O6 may be configured with an active axis that actively rotates the imaging units 21 and 21D around the axis according to the power of the actuator.

In the first to eighth embodiments described above, the order of processing of the flows illustrated in FIGS. 3, 10, and 13 may be changed within a consistent range. In addition, the techniques described in the first to eighth embodiments described above may be combined as appropriate.

The following configurations also belong to the technical scope of the present disclosure.
(1) A medical image processing device including:

a circuitry configured to

acquire a captured image obtained by capturing an image of a subject;

specify an area of interest among a plurality of image areas in the captured image; and

adjust a brightness index value which is an index of brightness for each pixel in the captured image in order to emphasize the area of interest in the plurality of the image areas with respect to other areas, wherein

the brightness index value is an index value used for controlling emission brightness of each light emitting element arranged in each of a plurality of divided areas of a display screen in a display device for displaying the captured image.
(2) The medical image processing device according to (1), wherein the circuitry is configured to adjust the brightness index value such that the brightness index value for each pixel in the other areas is darkened.
(3) The medical image processing device according to (1) or (2), wherein the circuitry is configured to

multiply the brightness index value for each pixel in the area of interest by a first multiplier, and

multiply the brightness index value for each pixel in the other areas by a second multiplier smaller than the first multiplier.
(4) The medical image processing device according to 1) or (2), wherein the circuitry is configured to multiply the brightness index value for each pixel in the other areas by a multiplier that decreases as a distance from the area of interest increases.
(5) The medical image processing device according to 1) or (2), wherein the circuitry is configured to multiply the brightness index value for each pixel in the plurality of the image areas by a multiplier that decreases as a distance from a center of the area of interest increases.
(6) The medical image processing device according to any one of (1) to (5), wherein the area of interest is a detection area for calculating an evaluation value used for at least one control of a first control for controlling a focal position of an imaging device that generates the captured image and a second control for controlling the brightness of the captured image.
(7) The medical image processing device according to any one of (1) to (6), wherein the area of interest is an area including an image center of the captured image.
(8) The medical image processing device according to any one of (1) to (5), wherein

the captured image is an image obtained by capturing fluorescence from the subject irradiated with excitation light, and

the area of interest is an area in which an intensity of a fluorescent component is a specific threshold value or more.
(9) The medical image processing device according to any one of (1) to (5), wherein

the captured image is obtained by capturing an image of the subject irradiated with light in a first wavelength band, and

the area of interest is an area corresponding to an area in which an intensity of a fluorescence component in a fluorescence image obtained by capturing fluorescence from the subject irradiated with the excitation light is a specific threshold value or more.
(10) The medical image processing device according to any one of (1) to (9), wherein the circuitry is further configured to execute enlargement processing of enlarging a specific area including the area of interest in the captured image, and

adjust the brightness index value for each pixel in an area other than the area of interest in the specific area so as to be darker than before the enlargement processing is executed, after the enlargement processing is executed. (11) The medical image processing device according to any one of (1) to (10), wherein the brightness index value is at least one of a Y value, a Cb value, and a Cr value for each pixel in the captured image.
(12) A medical observation system including:

the medical image processing device according to any one of (1) to (11); and

a display device configured to display the captured image processed by the medical image processing device, wherein the display device includes a plurality of light emitting elements arranged for each of a plurality of divided areas of the display screen and whose emission brightness is controlled according to the brightness index value.

According to a medical image processing device and a medical observation system according to the present disclosure, there is an effect that an image suitable for observation may be generated.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising:
a circuitry configured to
acquire a captured image obtained by capturing an image of a subject;
specify an area of interest among a plurality of image areas in the captured image; and
adjust a brightness index value which is an index of brightness for each pixel in the captured image in order to emphasize the area of interest in the plurality of the image areas with respect to other areas, wherein
the brightness index value is an index value used for controlling emission brightness of each light emitting element arranged in each of a plurality of divided areas of a display screen in a display device for displaying the captured image.

2. The medical image processing device according to claim 1, wherein the circuitry is configured to adjust the brightness index value such that the brightness index value for each pixel in the other areas is darkened.

3. The medical image processing device according to claim 1, wherein the circuitry is configured to
multiply the brightness index value for each pixel in the area of interest by a first multiplier, and
multiply the brightness index value for each pixel in the other areas by a second multiplier smaller than the first multiplier.

4. The medical image processing device according to claim 1, wherein the circuitry is configured to multiply the brightness index value for each pixel in the other areas by a multiplier that decreases as a distance from the area of interest increases.

5. The medical image processing device according to claim 1, wherein the circuitry is configured to multiply the brightness index value for each pixel in the plurality of the image areas by a multiplier that decreases as a distance from a center of the area of interest increases.

6. The medical image processing device according to claim 1, wherein the area of interest is a detection area for calculating an evaluation value used for at least one control of a first control for controlling a focal position of an imaging device that generates the captured image and a second control for controlling the brightness of the captured image.

7. The medical image processing device according to claim 1, wherein the area of interest is an area including an image center of the captured image.

8. The medical image processing device according to claim 1, wherein
the captured image is an image obtained by capturing fluorescence from the subject irradiated with excitation light, and
the area of interest is an area in which an intensity of a fluorescent component is a specific threshold value or more.

9. The medical image processing device according to claim 1, wherein
the captured image is obtained by capturing an image of the subject irradiated with light in a first wavelength band, and
the area of interest is an area corresponding to an area in which an intensity of a fluorescence component in a fluorescence image obtained by capturing fluorescence from the subject irradiated with the excitation light is a specific threshold value or more.

10. The medical image processing device according to claim 1, wherein the circuitry is further configured to execute enlargement processing of enlarging a specific area including the area of interest in the captured image, and
adjust the brightness index value for each pixel in an area other than the area of interest in the specific area so as to be darker than before the enlargement processing is executed, after the enlargement processing is executed.

11. The medical image processing device according to claim 1, wherein the brightness index value is at least one of a Y value, a Cb value, and a Cr value for each pixel in the captured image.

12. A medical observation system comprising:
the medical image processing device according to claim 1; and
a display device configured to display the captured image processed by the medical image processing device, wherein the display device includes a plurality of light emitting elements arranged for each of a plurality of divided areas of the display screen and whose emission brightness is controlled according to the brightness index value.
